(19) United States
(12) Patent Application Publication
(10) Pub. No.: US 2019/0184053 A1
(43) Pub. Date: Jun. 20, 2019

(54) Biaxially Transformed Sponges

(71) Applicant: Robert E. South, Warsaw, IN (US)

(72) Inventor: Robert E. South, Warsaw, IN (US)

(21) Appl. No.: 16/350,119

(22) Filed: Sep. 28, 2018

Related U.S. Application Data
(63) Continuation-in-part of application No. 15/005,161, filed on Jan. 25, 2016, now abandoned.

(51) Int. Cl. A61L 15/42; C08J 5/18

(52) U.S. Cl. CPC A61L 15/425; A61L 15/225; C08J 5/18

(57) ABSTRACT

A sheet sponge (10) of polyvinyl alcohol is anhydrously compressively transformed from an original shape, size, and thickness (FIGS. 1 and 2) into a second shape, size, and thickness (42) by curling (FIG. 3) or coiling (FIG. 4) buckling (FIG. 7), crumpling (FIG. 12), and layering crumpled portions (FIG. 12), on upon another, anhydrously compressively fusing the transformed sponge in the transformed shape, size, and thickness (FIG. FIGS. 7 and 8), and subsequently hydrating the transformed sponge back into its original shape, size, and thickness (FIGS. 1 and 2.

### BACKGROUND OF THE INVENTION

### Field of the Invention

The present invention relates generally to transforming the shape, size, and/or thickness of sheet sponges. More particularly, the present invention pertains to anhydrously biaxially transforming polyvinyl alcohol sheet sponges from a first shape, size, and thickness to a different size and thickness, compressively fusing the compressed sponges into their transformed shapes, and at a selected time and geographical location, hydrating the transformed sponges back to their original shapes and sizes.

### Description of the Related Art

Man-made sponges are used in many fields of endeavor and for many uses. They are used in the home, on the farm, in industry, in the medical field, and in the toy industry. Sponges are used for cleaning, filtering, shock absorbing, and sound absorbing. They are even used as advertising novelties.

Sponges now play a useful role in the cleaning and care of medical equipment. In the past, two layers of four inch (10.2 cm) gauze squares, dampened with water and a detergent, have been used to wipe down endoscopic insertion tubes. However, occasionally, lint from the gauze has entered the machine, causing functional problems and a need for additional cleaning. These problems can be obviated by using plastic sponges for wipes.

Presently, plastic-sponge wipes are available from N.M. Beale Company, Inc. in Harvard, Mass. Their plastic-sponge wipes are made from polyvinyl alcohol, are four inches (10.2 cm) in diameter, and are compacted to a thickness of 0.062 inches (1.58 mm). When hydrated, the sponges expand to a thickness of 0.125 inches (3.17 mm). The reason for compaction is to save space in the laboratory. thickness of 0.125 inches (3.17 mm). The reason for compaction is to save space in the laboratory.

### BRIEF SUMMARY OF THE INVENTION

In the present invention, a sponge is cut from a sheet of polyvinyl alcohol plastic foam of a given thickness, size and shape, is anhydrously transformed into a different size and a different thickness by biaxially compressing and compressive fusing, is transported to a place of use, and hydrated back into its original shape and shape at a selected time and place.

The original sheet sponge may be any suitable shape, size, and thickness. In like manner, the transformed shape may be of any suitable shape, size, and thickness. However, in a preferred embodiment, a sheet sponge that is rectangular in shape is transformed into a disk shape that is smaller and thicker than the original sheet sponge.

Obviously, when a rectangular shape is transformed into a disk shape that is smaller and thicker, both the perimeter and the area of the transformed shape are smaller than the perimeter and area of the original shape.

Subsequent to anhydrously transforming a sheet sponge into a shape that is smaller and thicker is hydrated, at a selected geographical location and at a selected time, the transformed sponge is hydrated back to its original shape and size. That is, if has been anhydrously transformed from a rectangular sheet to a disk, the hydrated sponge transforms back to the size of its original rectangular shape and to its original thickness.

In a first aspect of the present invention, a method comprises: producing a polyvinyl alcohol sheet sponge that comprises an original shape, size, and thickness; anhydrously compressively transforming the sheet sponge to a smaller size and larger thickness; anhydrously compressively fusing the transformed sponge in the smaller size and larger thickness; and hydrating the fused sponge back to the original shape, size, and thickness.

In a second aspect of the present invention, a method comprises: producing a polyvinyl alcohol sheet sponge that comprises an original shape, size, and thickness; crumpling the sheet sponge; layering portions of the crumpled sheet sponge, one on another; anhydrously compressively fusing the layered sponge; and hydrating the fused sponge back to the original shape, size, and thickness.

In a third aspect of the present invention a method comprises: producing a polyvinyl alcohol sheet sponge that comprises an original shape, size, and thickness; anhydrously compressively transforming the sheet sponge to a smaller size and larger thickness; the anhydrously compressively transforming comprises crumpling the sheet sponge and layering portions of the crumpled sponge, one on another; anhydrously compressively fusing the transformed sponge in the smaller size and larger thickness; and hydrating the fused sponge back to the original shape, size, and thickness in less than 30 seconds in water at 70 degrees Fahrenheit (21 degrees Celsius).

### BRIEF DESCRIPTION OF SEVERAL VIEWS OF THE DRAWINGS

FIG. 1 is a plan view of a plastic sponge with a rectangular shape that includes four edges and a center;

FIG. 2 is an end view of the plastic sponge of FIG. 1, taken substantially as shown by view line 2-2 of FIG. 1;

FIG. 3 is an end view of the plastic sponge of FIG. 1 subsequent to curling around an axis;

FIG. 4 is an end view of the plastic sponge of FIG. 1 subsequent to coiling around an axis;

FIG. 5 is a cross-sectioned front elevation of a die set, with the coiled sponge of FIG. 4 inserted axially into a cylindrical die bore of the die set, and with a die plunger axially inserted into the cylindrical die bore for longitudinal movement against the coiled sponge;

FIG. 6 is a top view of the die set of FIG. 5 with the die plunger of FIG. 5 removed to reveal the coiled sponge of FIG. 4;

FIG. 7 is a cross-sectioned elevation of the die set of FIG. 5, taken substantially as shown in FIG. 5, except that the die plunger has moved one edge of the coiled sponge downwardly toward another edge causing the coiled sponge to randomly buckle in two places;

FIG. 8 is a top view of the coiled sponge of FIG. 4 after it has been crumpled, layered, anhydrously compressed, and anhydrously fused into a disk shape by the die set of FIGS. 5-7;

FIG. 9 is a front elevation of the disk-shaped sponge of FIG. 8, taken substantially as shown by view line 9-9 of FIG. 8;

FIG. 10 is a front elevation, taken substantially the same as FIG. 9, of a partially hydrated sponge of the present invention, illustrating transformation of the disk-shaped sponge of FIGS. 8 and 9 as it begins to hydrate back into the original shape, size, and thickness of the rectangular-shaped sponge of FIGS. 1 and 2;

FIG. 11A is a top view, taken substantially the same as FIG. 3, showing two sponges curled together;

FIG. 11B is a top view, taken substantially the same as FIG. 3, showing two sponges curled serially;

FIG. 11C is a top view, taken substantially the same as FIG. 4, showing two sponges coiled together;

FIG. 11D is a top view, taken substantially the same as FIG. 4, showing two sponges coiled serially; and

FIG. 12 is a front elevation of the sheet sponge 10 of FIGS. 1 and 3, taken substantially the same as the randomly buckled sponge of FIG. 7, and also taken substantially the same as the partially hydrated sponge of FIG. 10, illustrating both crumpling of the random buckled portions of FIG. 7 and layering of the crumpled portions, one upon another, as the plunger 32 of FIG. 7 moves farther into the bore 28.

### DETAILED DESCRIPTION OF THE INVENTION

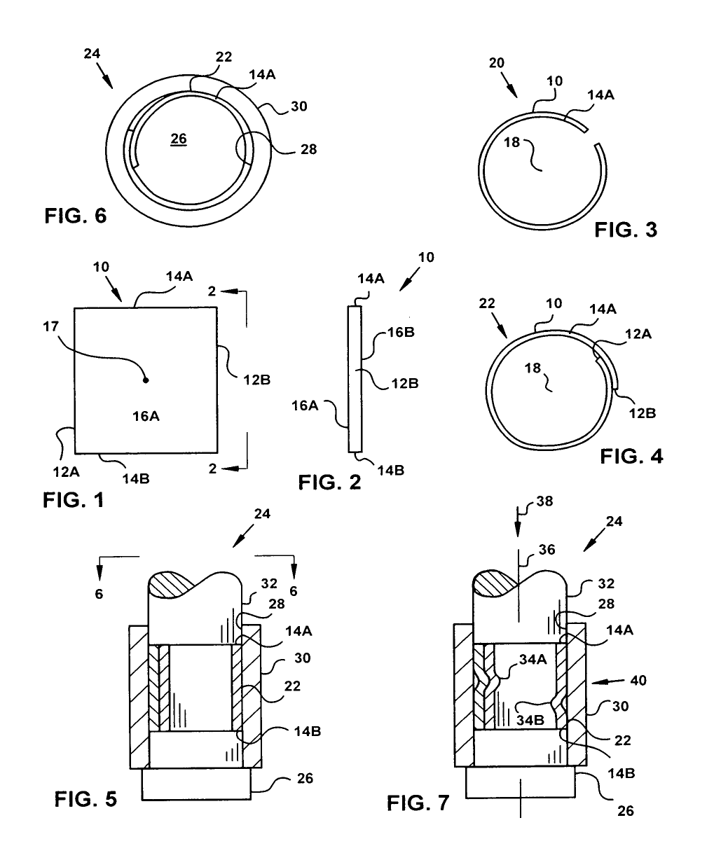

Referring now to FIGS. 1 and 2, a sheet sponge 10 has a rectangular shape and includes opposing edges 12A and 12B, opposing edges 14A and 14B, surfaces 16A and 16B, and a center 17. The rectangular-shaped sponge 10 preferably is 4.0 inches (10.2 cm) square, and is 0.125 inches (3.17 mm) thick.

The sponge material that Applicant uses to practice the present invention is polyvinyl alcohol and requires a compression pressure of 2.8 pounds per square inch for twenty-five percent compression. In contrast, typical open cell polyurethane requires only 0.33 pounds per square inch to compress twenty percent. Further, Applicant's polyvinyl alcohol sponge material has a density of 2.8 pounds per cubit foot, whereas open cell polyurethane typically has a density of only 1.8 pounds per cubic foot.

While polyvinyl alcohol is an accurate name for the preferred chemical compound used in the present invention, polyvinyl alcohol is also known as polyvinyl acetal. Methods for making polyvinyl alcohol are taught by Wilson, in U.S. Pat. No. 2,609,347 which issued on Sep. 2, 1952, and by Takahashiet et al., in U.S. Pat. No. 3,673,125 which issued on Jun. 27, 1972.

Applicant compresses his polyvinyl alcohol sponges at 2,800 pounds per square inch. This reduces the volume of the cleansing sponges to approximately eleven percent of their original volume.

Referring now to FIGS. 1-3, the sponge 10 of FIGS. 1 and 2 has been curled around an axis 18 thereby forming a curled sponge 20 of FIG. 3 with the edge 14A of FIGS. 1 and 2 becoming a curled or coiled edge 15A of FIG. 3. As shown in FIG. 3, the curled sponge 20 extends only partially around the axis 18.

Referring now to FIGS. 1, 2, and 4, the sponge 10 of FIGS. 1 and 2 has been coiled around the axis 18 thereby forming a coiled sponge 22 with a curled or coiled edge 15A shown on FIGS. 4 and 5, and a curled or coiled edge 15B shown on FIG. 5.

As defined herein, a curled sponge, such as the curled sponge 20 of FIG. 3, is one in which the edges 12A and 12B do not overlap circumferentially. Further, as defined herein, a coiled sponge is one, such as the coiled sponge 22 of FIG. 4, in which the edges 12A and 12B overlap circumferentially.

Referring now to FIGS. 5 and 6, a die set 24 includes a plug 26 that is disposed into a cylindrical die bore 28 of a die body 30, and a cylindrical die plunger 32 that is slidably inserted into the die bore 28. Preferably the die bore 28 has a diameter of 1.25 inches (3.17 mm).

As shown in FIG. 5, the coiled sponge 22 has been inserted axially into the die 28, and the die plunger 32 has been inserted into the die bore 28. As shown in FIG. 5, the die plunger 32 is touching the curled or coiled edge 15A of the coiled sponge 22, but the die plunger 32 has not yet moved the curled or coiled edge 15A toward the curled or coiled edge 15B.

While use of a coiled sponge, such as the coiled sponge 22, is preferred, optionally, a curled sponge, such as the curled sponge 20 of FIG. 3 may be used in the die set 24.

Referring now to FIG. 7, the die set 24 of FIG. 5, with all of its parts, is shown in FIG. 7, and the die plunger 32 has moved downwardly into the die bore 28, moving the curled or coiled edge 15A of the coiled sponge 22 downward, closer to the curled or coiled edge 15B, thereby producing buckled portions 34A and 34B, and with further movement of the plunger 32 into the bore 28, the coiled sponge 22 with buckled portions 34A and 34B, will be transformed into a crumpled sponge, and the crumpled sponge will be transformed into the crumpled and layered sponge of FIG. 12.

A curved or coiled piece of paper that is subjected to a column load will buckle, crumple, and fold into a plurality of layers. In like manner, the coiled sponge 22 buckles randomly in a plurality of places, crumples, and folds into a plurality of layers as the plunger 32 moves downward, as shown and numbered in FIG. 12.

Buckling of the coiled sponge 22 in the die set 24 is random, varying at different points longitudinally and circumferentially with respect to the die bore 28, and varying from piece to piece. Therefore, the buckled portions 34A and 34B in FIG. 7 are primarily illustrative.

The process described in conjunction with FIGS. 5-7 moves the curled or coiled edge 15A toward the curled or coiled edge 15B along a longitudinal axis 36 of FIG. 7, as shown by a longitudinal arrow 38.

Anhydrously compressively transforming the sponge 10 into a shape that is smaller and thicker comprises forcing an edge 14A of FIG. 1 closer to a center 17, and forcing edges 14A and 14B of the sheet sponge 10 closer together. With respect to movement of the plunger 32 into the bore 28, anhydrously compressively transforming the sponge 10 comprises curling or coiling the sponge 10 and forcing curled or coiled edges 15A and 15B closer together.

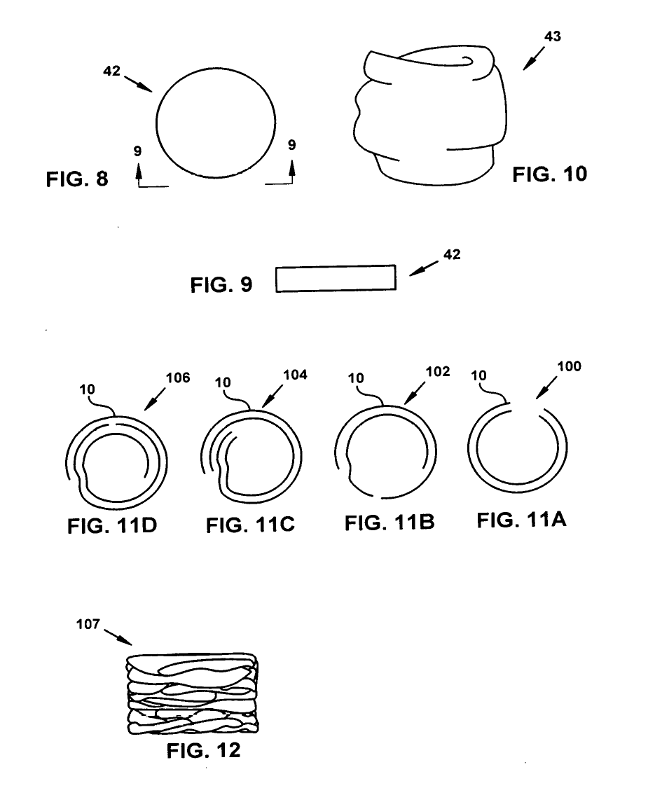

Movement of the plunger 32 into the bore 28 moves portions of the coiled sponge 22 radially, as shown by a transverse arrow 40 to fill an opening 23 inside the coiled sponge 22, to form a disk-shaped sponge, or biaxially transformed sponge, 42 of FIGS. 8 and 9.

Therefore, the present process transforms the coiled sponge 22 biaxially. That is, the coiled sponge 22 is transformed with regard to one axis by moving the edge 14A toward the edge 14B, as illustrated by the arrow 38; and the coiled sponge 22 is transformed with regard to another axis as portions of the coiled sponge 22 are moved and/or folded radially, as illustrated by the arrow 40, to fill the opening 23 and form the disk-shaped sponge, or disk 42.

Referring now to FIGS. 8 and 9, the process, as particularly described in conjunction with FIG. 7, results in the disk-shaped sponge, or disk 42. The sponge 10 of FIGS. 1 and 2 that was 4.0 inches (10.2 cm) square has been biaxially transformed into the disk-shaped sponge 42 of FIGS. 8 and 9.

The disk-shaped sponge, or disk 42 of FIGS. 8 and 9 has a diameter of approximately 1.29 inches (3.28 mm) and a thickness of approximately 0.165 inches (4.19 mm). The rectangular-shaped sponge 10, which had an original volume of 2.0 cubic inches (32.8 cubic cm), has been reduced to 0.216 cubic inches (3.54 cubic cm) in the disk-shaped sponge, or disk 42. Therefore, the volume of the rectangular-shaped sponge 10 has been reduced by 89.2 percent in the disk-shaped sponge 42.

In contrast, sponges previously used for medical use, before compacting, were four inches diameter. This diameter is too large to design a compact dispenser, and the sponge was too soft for reliable dispensing. After compacting a thickness of 0.125 inch (3.17 mm) to a thickness of 0.062 inches (1.58 mm), the four inch (10.2 cm) diameter sponges remained too large to make a compact dispenser. After compacting, a new problem for dispensing had been introduced—the compacted sponge was far from flat.

However, when biaxially transformed as taught herein, the disk shaped sponge is smaller and flatter than previously available cleansing sponges. Therefore, advantages of biaxially transforming sponges for medical use include compactness, the ability to sell and distribute the sponges in small-diameter tubes, the ability to design compact dispensers, and ease of dispensing the sponges individually.

With regard to using, the present invention provides a polyvinyl alcohol sheet sponge that hydrates more rapidly, thereby saving the technician's time. More particularly, the disk-shaped sponge 42 completely hydrates in less than 30 seconds with water at 70 degrees Fahrenheit (21 degrees Celsius).

In contrast, when the polyvinyl alcohol sponge is used for medical purposes, it is 4.0 inches (10.2 cm) in diameter, and has been compressed from 0.125 inches (3.17 mm) to 0.0625 inches (1.58 mm). In water that is 70 degrees Fahrenheit (21 Celsius), the prior art sponge sometimes is not fully hydrated in 60 seconds, and may require massaging to finish hydrating.

Referring now to FIG. 10, a partially-hydrogenated sponge 43 is illustrative of the disk sponge 42 of FIGS. 8 and 9 as it transforms back into the rectangular-shaped sponge 10 of FIGS. 1 and 2.

Referring now to FIGS. 11A, 11B, 11C, and 11D, these drawings are top views, taken substantially the same as FIGS. 3 and 4.

In FIG. 11A, curled sponges 100 include two sponges 10 of FIG. 1 curled together as if two of the sponges 10 were stacked together and then curled.

In FIG. 11B, curled sponges 102 include two sponges 10 of FIG. 1 that are curled serially. It is as if one of the sponges 10 were curled and then an other of the sponges 10 were used to continue the curl. It is as if a single sponge were curled.

In FIG. 11C, coiled sponges 104 include two sponges 10 of FIG. 1 that are coiled together. That is, it is as if two of the sponges 10 were stacked together and then coiled.

In FIG. 11D, coiled sponges 106 include two of the sponges 10 of FIG. 1 that are coiled serially.

Referring now to FIG. 12, a partially-transformed sponge 108 with crumpling portions 110A and 110B folded into layered portions 112A and 112B, so that the partially-transformed sponge 108 comprises a plurality of randomly layered portions.

That is, as the plunger 32 of FIG. 5 continues to move into the bore 28, the coiled sponge 22 of FIG. 4 with the random buckled portions 34A and 34B of FIG. 7, the sponge, 20 or 22, is biaxially anhydrously compressively transformed into the partially-transformed sponge 108 with the crumpling portions 110A and 110B folded into the layered portions 112A and 112B.

Random crumpling and layering can best be understood by comparing the cross-sectional area of the sponge 10 with the cross-sectional area of the bore 28. Since the sponge 10 of FIGS. 1 and 2 is four inches (10.2 cm) square and has a thickness of 0.125 inches (3.17 mm), an area of the end 14A is 0.50 square inches (3.243 square cm). And since the bore 28 has a diameter of 1.125 inches (28.6 mm), it has a cross-sectional area of 0.994 square inches (6.41 cm). That is, the cross-sectional area of the bore 28 is twice that of the sponge 10.

When the curled sponge 20 of FIG. 3, which has a length of four inches (10.2 mm), is pressed into the bore 28 with a cross-sectional area that is twice as large as the cross-sectional area of the sheet sponge 10, random crumpling and layering will occur.

If we were to assume that the sponge 10 were crumpled completely into the bore 28, and became perfectly conformed to the bore 28, before compression of the sponge 10 begins, the plunger 32 would have traveled two inches (5.08 cm) into the bore 28.

Even though it is not reasonable to assume that complete crumpling occurs before compression starts, and even though some crumpling occurs simultaneously with compressing, buckling and crumpling both recite steps that are performed before compressively transforming the sheet sponge into the disk 42 of FIGS. 8 and 9.

In summary, since, as shown in FIG. 7, the process of the present invention moves the edge 14A in FIG. 7 toward the edge 14B, as shown by the longitudinal arrow 38, and also moves the portions 34A and 34B of the coiled sponge 22 radially inward as shown by the transverse arrow 40, it can be seen that the present process transforms any sheet sponge, such as the sheet sponge 10 biaxially.

An objective of the present invention is to facilitate shipment, storing, and especially dispensing of cleansing sponges. Prior to the present invention, cleansing sponges were four inch (10.2 mm) squares, 0.062 inches (1.58 mm) thick, and had a flatness resembling randomly-curved potato chips.

Because of their large size and their large departure from flatness, these cleansing sponges were shipped in plastic bags, much like potato chips bags, but larger, stored in the large bags, and dispensed from the large bags, one at a time, as needed.

An other object of the present invention is to provide a method for anhydrously transforming cleansing sponges 10 into disk-shaped sponges 42 that are smaller in diameter, thicker, and flatter, so that they can be shipped in compact tubes, stored in compact tubes, dispensed from a tube on the laboratory counter, and even dispensed from a machine.

As taught throughout this specification, the entire process is performed without water and without any binder. That is, the entire process is anhydrous. Sheet sponges, after they are anhydrously curled, crumpled, biaxially compressed, and folded into layers, are anhydrously fused in a transformed form.

While, as set forth above, the objective of the present invention was to change the size and shape of cleansing sponges, subsequently, an unintentional advantage was discovered: sponges that are biaxially compressed, as taught herein, hydrate faster than sponges that are compressed along a single axis. As taught above, cleansing sponges that are biaxially compressed as taught herein hydrate in half of the time required for previously-available cleansing sponges.

Watching biaxially-compressed sponges of the present invention change their shape as they hydrate reveals why they hydrate more rapidly than single-axis compressed sponges. In the process of hydrating, they release energy that was trapped by fusing contacting portions of the crumpled sponge. Cleansing sponges that have been biaxially compressed as taught herein appear to be stretching and straightening themselves as crumpled portions straighten out. The fact that they change their shape while hydrating proves that they are releasing trapped energy.

The method of the present invention comprises anhydrously compressively transforming a sheet sponge from an original shape, size, and thickness to a second size and thickness; storing energy of the transforming step in the sponge; releasing the stored energy; the releasing step comprises hydrating the sheet sponge in water; and the restoring step further comprises restoring the sheet sponge to the original shape, size, and thickness.

Alternately, or in combination, the method of the present invention comprises: producing a polyvinyl alcohol sheet sponge that comprises an original shape, size, and thickness; anhydrously compressively transforming the sheet sponge to a smaller size and larger thickness; anhydrously compressively fusing the transformed sponge in the smaller size and larger thickness; and hydrating the fused sponge back to said original shape, size, and thickness.

Anhydrously compressively transforming comprises curling the sheet sponge, moving an edge of the curled sponge toward a center, crumpling the curled sponge, and layering portions of said crumpled sponge, one upon another.

Alternately, anhydrously compressively transforming comprises moving one edge of a polyvinyl alcohol sheet sponge toward another edge of the sheet, or moving one edge of a polyvinyl alcohol sheet sponge toward a center of the sheet, crumpling the sheet sponge; and layering crumpled portions of said sheet sponge, one upon another.

Preferably, the producing step comprises producing a polyvinyl sheet sponge with a density of 2.8 pounds per cubic foot. Preferably, both anhydrously compressively transforming and anhydrously compressively fusing comprises applying a pressure of 2,800 pounds per square inch; and reducing a volume of said sheet sponge by 89.2 percent. And hydrating comprises hydrating the fused sponge back to the original shape, size, and thickness in less than 30 seconds in water at 70 degrees Fahrenheit (21 degrees Celsius).

The method comprises: producing a polyvinyl alcohol sheet sponge that comprises an original shape, size, and thickness; crumpling the sheet sponge; layering portions of the crumpled sheet sponge, one on another; anhydrously pressure fusing the layered sponge; and hydrating the fused sponge back to the original shape, size, and thickness. Alternately, or in combination, the method comprises coiling the sheet sponge; inserting the coiled sponge into a bore; and performing the crumpling and layering steps in the bore.

With regard to the producing step recited in the claims, as defined in one dictionary, and as defined herein, producing alternately means bringing forth or bringing into existence.

It should be abundantly clear, even those who are not skilled in the art, that the producing step alternately comprises bringing into existence by producing PVC sheets as taught by the referenced patents and subsequently cutting into pieces of the desired size and shape, bringing forth by buying rolls of PVC sheet material and cutting into pieces with the desired size and shape, and bringing forth by purchasing pieces of PVC sheets already cut to the desired size and shape.

Clearly, the present invention pertains to a method for anhydrously transforming sheets of plastic into a smaller and thicker form, and for hydrating the transformed sheets back into the original size and shape, not whether the material to be transformed is brought into existence or brought forth.

As defined herein, a shape is three dimensional, and has a volume. A sponge that is rectangular in shape has a first volume, even though its thickness may be only one-thirtieth of its width or length. Therefore, a rectangular-shaped sponge has a volume, and is biaxially transformed into a disk-shaped sponge, or disk, that has a second and smaller volume.

Further, as defined herein, a curled sponge, such as the curled sponge 20 of FIG. 3, is generic to both curled and coiled sponges.

While specific apparatus and method have been disclosed in the preceding description, it should be understood that these specifics have been given for the purpose of disclosing the principles of the present invention, and that many variations thereof will become apparent to those who are versed in the art.

What is claimed is:

1. A method which comprises:
a) producing a polyvinyl alcohol sheet sponge that comprises an original shape, size, and thickness;
a) anhydrously compressively transforming said sheet sponge to a smaller size and larger thickness;
b) anhydrously compressively fusing said transformed sponge in said smaller and size and larger thickness; and
c) hydrating said fused sponge back to said original shape, size, and thickness.

2. The method as claimed in claim 1 in which said anhydrously compressively transforming comprises curling said sheet sponge.

3. The method as claimed in claim 1 in which said anhydrously compressively transforming comprises crumpling said sheet sponge.

4. The method as claimed in claim 1 in which said anhydrously compressively transforming comprises layering portions of said sheet sponge, one upon another.

5. The method as claimed in claim 1 in which said anhydrously compressively transforming comprises:
a) crumpling said sheet sponge; and
b) layering portions of said crumpled sponge, one upon another.

6. The method as claimed in claim 1 in which said anhydrously compressively transforming comprises
a) curling said sheet sponge;
b) crumpling said curled sponge; and
b) layering portions of said crumpled sponge, one upon another.

7. The method as claimed in claim 6 in which:
a) said producing step comprises producing a polyvinyl sheet sponge with a density of 2.8 pounds per cubic foot; and
b) said anhydrously pressure transforming comprises applying a pressure of 2,800 pounds per square inch; and reducing a volume of said sheet sponge by 89 percent.
c) said hydrating comprises hydrating said fused sponge back to said original shape, size, and thickness in less than 30 seconds in water at 70 degrees Fahrenheit (21 Celsius).

8. The method as claimed in claim 1 in which:
a) said anhydrous transforming comprises selecting a polyvinyl sponge with a density of 2.8 pounds per cubic foot; and
b) said hydrating comprises hydrating said fused sponge back to said original shape, size, and thickness in less than 30 seconds in water at 70 degrees Fahrenheit (21 Celsius).

9. The method as claimed in claim 1 in which said sheet sponge comprises an edge and a center, and said anhydrously compressively transforming comprises forcing said edge toward said center.

10. The method as claimed in claim 1 in which said sheet sponge comprises an edge and a center, and said anhydrously compressively transforming comprises:
a) forcing said edge toward said center; and
b) said forcing step comprises layering portions of said sheet sponge, one on another.

11. The method as claimed in claim 10 in which:
a) said producing step comprises producing a polyvinyl sheet sponge with a density of 2.8 pounds per cubic foot; and
b) said anhydrously pressure transforming comprises applying a pressure of 2,800 pounds per square inch; and reducing a volume of said sheet sponge by 89 percent; and
c) said hydrating comprises hydrating said fused sponge back to said original shape, size, and thickness in less than 30 seconds in water at 70 degrees Fahrenheit (21 Celsius).

12. A method which comprises:
a) producing a polyvinyl alcohol sheet sponge that comprises an original shape, size, and thickness;
b) anhydrously compressively transforming said sheet sponge to a smaller size and larger thickness;
c) said anhydrously compressively transforming comprises crumpling said sheet sponge and layering portions of said crumpled sponge, one on another;
d) anhydrously compressively fusing said transformed sponge in said smaller and size and larger thickness; and
e) hydrating said fused sponge back to said original shape, size, and thickness in less than 30 seconds in water at 70 degrees Fahrenheit (21 Celsius).

13. The method as claimed in claim 12 in which said anhydrously compressively transforming comprises:
a) coiling said sheet sponge; and
b) inserting said sheet sponge into a bore prior to said crumpling and layering steps.

14. The method as claimed in claim 12 in which:
a) said producing step comprises producing a polyvinyl sheet sponge with a density of 2.8 pounds per square inch; and
b) said anhydrously pressure transforming comprises applying a pressure of 2,800 pounds per square inch; and reducing a volume of said sheet sponge by 89 percent.

15. The method as claimed in claim 12 in which said producing step comprises producing a polyvinyl sheet sponge with a density of 2.8 pounds per cubic foot.

16. A method which comprises:
a) anhydrously compressively transforming a sheet sponge into a disk;
b) anhydrously compressively fusing said disk;
c) said transforming and fusing steps comprise curling said sheet sponge, inserting said curled sheet sponge axially into a cylindrical die bore, and forcing a die plunger into said die bore;
d) transforming said fused disk into a hydrated sheet sponge; and
e) the second said transforming step comprises hydrating said disk.

17. The method as claimed in claim 16 in which said anhydrously compressively transforming and fusing steps comprise crumpling said sheet sponge and anhydrously compressively fusing portions of said sheet sponge, one upon another.

18. The method as claimed in claim 16 in which said anhydrously compressively transforming and fusing steps comprise applying a pressure of about 2,800 pounds per square inch with said die plunger.

19. The method as claimed in claim 16 in which said anhydrously compressively transforming step comprises reducing a volume of said sheet sponge by about 89 percent.

20. The method as claimed in claim 16 in which said hydrating step comprises hydrating said fused disc into said hydrated sheet sponge in less than 30 seconds in water at 70 degrees Fahrenheit (21 Celsius).
